(19) United States
(12) Patent Application Publication
(10) Pub. No.: US 2012/0178128 A1
(43) Pub. Date: Jul. 12, 2012

(54) PROCESS FOR PRODUCING CYSTEINE AND/OR GLUTATHIONE FROM CYSTINE EMPLOYING YEAST

(75) Inventor: Bertus Noordam, 's-Gravenzande (NL)

(73) Assignee: DSM IP ASSETS B.V., Heerlen (NL)

(21) Appl. No.: 13/496,021

(22) PCT Filed: Sep. 28, 2010
(86) PCT No.: PCT/EP2010/064309

§ 371 Date: Mar. 14, 2012

(30) Foreign Application Priority Data
Sep. 29, 2009 (EP) 09171678.7

(51) Int. Cl. C12P 21/02; A23L 1/28; C12P 13/12

(52) U.S. Cl. 435/71.1; 435/113; 426/655

(57) ABSTRACT

The invention provides a process for the conversion of cystine to cystein and/or glutathione comprising contacting cystine with a microorganism. The invention also relates to a yeast extract comprising at least 1.8 mg/g cystein and a yeast autolysate comprising at least 1.3 mg/g cystein.

### FIELD OF THE INVENTION

This invention relates to a process for the production of cystein and/or glutathione from cystine.

### BACKGROUND OF THE INVENTION

A major source of commercially available cystein is cystine. Cystine can be electrochemically reduced to cystein (sometimes spelled “cysteine”). This process is expensive and complex. JP03180188 describes a method to produce cystein from cystine in alkaline conditions using a solution with an alkali-resistant enzyme with hydrogenase activity. JP02092294 describes a method to produce cystein from cystine using an enzyme solution with hydrogenase activity.

### DESCRIPTION OF THE INVENTION

Surprisingly, we have found that cystein and/or glutathione may be produced by contacting cystine with a microorganism. In a first aspect the invention therefore provides a process for the production of cystein and/or glutathione from cystine comprising contacting cystine with a microorganism and recovering the cystein and/or glutathione.

In a preferred embodiment the temperature of the process of the invention is 0-100° C. Preferably the temperature is between 20-80° C., more preferably between 30-70° C., even more preferably between 35-65° C., most preferably between 40-60° C. If the temperature is too low (i.e. below 0° C.) the conversion from cystine to cystein and/or glutathione may not take place or only occur to a little extent. Moreover, at low temperature the solubility of cystine is low, which may result in too little cystine in solution. If the temperature is too high (i.e. above 100° C.) the conversion from cystine to cystein and/or glutathione may not take place or only occur to a little extent.

Microorganisms may store cystein intracellular in the form of glutathione. Therefore, during the contacting of cystine with a microorganism, not only cystein, but also glutathione may be formed. Depending on the stage in the process of the first aspect of the invention, only cystein, only glutathione, or a combination of both may be detected. Production of glutathione implies that cystein was also produced but was converted to glutathione.

The pH of the process of the invention is preferably between 2 and 10. Preferably the pH is between 3 and 9, more preferably between 4 and 8, even more preferably between 5 and 7.

In a preferred embodiment the process of the invention is done on industrial scale. Throughout the description of the invention, an industrial scale process or an industrial process may be understood to encompass a process on a volume scale which is ≧10 L, preferably ≧100 L, more preferably ≧1 m³, ≧5 m³, even more preferably ≧10 m³, most preferably ≧25 m³, preferably less than 250 m³.

Any type of microorganism may be used in the process of the invention. Preferably the microorganism is suitable for the conversion of cystine to cystein and/or glutathione. Bacterial and fungal microorganisms are preferred, such as those which are suitable for food and feed applications. Preferred microorganisms are those that have the status of being food-grade and thus can be safely applied to food for human consumption. Examples of microorganisms suitable to be used in the process of the invention include filamentous fungi, such as Trichoderma or Aspergillus, and yeast. Preferably the microorganism is yeast. Yeast strains belonging to the genera Saccharomyces, Kluyveromyces or Candida are preferably used. Yeast strains belonging to the genus Saccharomyces, for example to the strain Saccharomyces cerevisiae are even more preferred. Examples of suitable bacterial microorganisms are Clostridia, Escherichia, and Archaea such as Methanobacterium and Methanosarcina.

In a preferred embodiment the microorganism in the process of the invention is in a fermentation media. The cystine may be contacted with a living (live) microorganism, which may result in the formation of cystein and/or glutathione in the fermentation media, or it may result in a microorganism (i.e. a microbial cell) with an increased cystein and/or glutathione content as compared to the microorganism obtained in a fermentation in which no cystine is present and/or to which no cystine has been added. Preferably the cystine is added to the fermentation media, preferably at the start of the fermentation. The fermentation may be done using a rich media, for example containing yeast extract of protein hydrolysate, but may also be a defined or minimal media. A defined or minimal media may have the advantage that the media composition can be defined such that the microorganism is stimulated to convert cystine to cystein and/or glutathione, for example by omitting sulphur sources other than cystine, or by keeping the concentration of such sulphur source as low as possible. For example, it may be advantageous to add no sulphates (sulphate salts) to the fermentation media. A defined or minimal media may comprise the usual vitamins, cofactors, and trace elements which are required for growth. The skilled person knows what vitamins, cofactors, and trace elements are required for growth for different types of microorganisms, but they are also listed in fermentation or microbiological hand books and can thus easily be found by the skilled person without undue burden.

In another embodiment the cystine may be contacted with a killed but intact microorganism. For example, the cystine may be contacted with killed but intact microorganism at the end of a fermentation, for example after killing-off. A killed but intact microorganism may be obtained by applying a heat shock, for example at 90-100 C.°, which may result in permeable (“leaky”) cells.

In yet another embodiment the cystine may be contacted with a lysed microorganism. For example, the cystine may be contacted with a lysed microorganism during the production of yeast extract. A lysed organism consists of a cell wall fraction (cell walls) and a soluble fraction. Using cell walls as a lysed microorganism may be advantageous in that they may be isolated from the cystein and/or glutathione after the process of the invention and may be re-used. A lyzed microorganism may be obtained by treating mechanically, chemically, or enzymatically. Mechanical treatments include homogenisation techniques. At this purpose, use of high-pressure homogenisers is possible. Other homogenisation techniques may include mixing with particles, e.g. sand and/or glass beads, or the use of a milling apparatus (e.g. a bead mill). The treatment may also be done by heating the cell. Chemical treatments include the use of salts, acid or alkali and/or one or more surfactants or detergents. Chemical treatments are less preferred because they may lead to degradation or modification of cystein or glutathione. Enzymatic treatments may be done using cellulases, glucanases, hemicellulases, chitinases, proteases and/or pectinases. A combination of treatments is also possible.

In a preferred embodiment the lysed organism are cell walls (e.g. in the form of cell walls). The cell walls may be part of a yeast autolysate, but may also be separated from released cell contents.

In a preferred embodiment a reductant and/or a cofactor is present in the process of the invention. Adding a reductant and/or a cofactor may enhance the rate and/or conversion and/or yield of the process. The reductant and/or cofactor is preferably selected from the group consisting of (reduced) nicotinamide adenine dinucleotide, (reduced) nicotinamide adenine dinucleotide phosphate, (reduced) lipoamide, (reduced) flavin adenine dinucleotide (FAD), (reduced) flavin mononucleotide (FMN), (reduced) metal ions, and H₂.

In another embodiment one or more enzymes are present in the process of the invention. The enzyme is preferably selected from the group consisting of protease, cystein reductase, hydrogenase, and lipoamide dehydrogenase. Preferably the one or more enzymes are added to the microorganism.

In an embodiment a cofactor regeneration system is present. Such a system may consist of an enzyme, usually an oxido-reductase, together with a substrate which can be oxidized by the enzyme. Examples of cofactor regeneration systems are glucose dehydrogenase/glucose and formate dehydrogenase/formate. A cofactor regeneration system may be required for the conversion of cystine to cystein or glutathione, or it may increase or enhance said conversion, or it may result in a process which proceeds for a longer time.

In another aspect the invention provides a yeast extract rich in cystein and/or glutathione, preferably comprising at least 1.8 mg/g cystein based on total dry matter. The Food Chemical Codex defines a “yeast extract” as follows: “Yeast Extract comprises the water soluble components of the yeast cell, the composition of which is primarily amino-acids, peptides, carbohydrates and salts. Yeast extract is produced through the hydrolysis of peptide bonds by the naturally occurring enzymes present in edible yeast or by the addition of food-grade enzymes”. Cystein and glutathione are major sources for the preparation of process flavours by reacting with reducing saccharides. U.S. Pat. No. 4,592,917 describes the preparation of a boiled chicken flavour by reacting a reducing saccharide with an amino acid (leucine) and a sulphur-containing substance which may be cystein. Yeast extract has been known for many years as a source of protein, peptides, aminoacids such as cystein, fats, minerals and B-vitamins. A yeast extract rich in cystein would be a suitable cystein or glutathione source for producing process flavours. Preferably the amount of cystein in the yeast extract of the invention is at least 4.6 mg/g, 4.7 mg/g, more preferably at least 5 mg/g, 5.1 mg/g, 6.1 mg/g, 6.2 mg/g, even more preferably at least 6.7 mg/g, 6.8 mg/g based on total dry matter. Preferably the yeast extract of the invention does not comprise any added glutathione or cystein.

In a preferred embodiment the yeast extract of the invention comprises at least 1% w/w 5′-ribonucleotides based on NaCl free dry matter weight. 5′-ribonucleotides, especially 5′-IMP and 5′-GMP, are known for their flavour enhancing properties. They are capable of enhancing the savoury and delicious taste in certain types of food. This phenomenon is described as ‘mouthfeel’ or umami. Yeast extracts rich in 5′-ribonucleotides are usually added to soups, sauces, marinades and flavour seasonings. Preferably the amount of 5′-ribonucleotides in the yeast extract of the invention is at least 2% w/w, 3%, 4%, more preferably at least 6, 8, 10% w/w, even more preferably at least 12%, 14%, 16%, even more preferably at least 18%, 20%, 22%, most preferably at least 25% w/w based on NaCl free dry matter weight. The weight percentage of 5′-ribonucleotides in the yeast extract of the invention (% w/w) is based on the weight of the NaCl free dry matter of the composition and is calculated as disodium salt heptahydrate (2Na.7Aq) of 5′-ribonucleotide. NaCl free does not mean that the yeast extract cannot contain NaCl, but means that NaCl is excluded from the yeast extract for the calculation of % w/w. The latter calculation can be performed by methods known to those skilled in the art.

In another aspect the invention provides a yeast autolysate rich in cystein and/or glutathione, preferably comprising at least 1.2 mg/g w/w cystein based on total dry matter. The Food Chemical Codex defines Autolysed Yeast as follows: “Autolysed Yeast is the concentrated, not extracted, partially soluble digest obtained from food-grade yeasts. Solubilisation is accomplished by enzyme hydrolysis or autolysis of yeast cells. Autolysed Yeast contains both soluble and insoluble components derived from the whole yeast cell'. A yeast autolysate differs from the “yeast extract” because the yeast autolysate, in addition to all the interesting components present in yeast extracts, also contains interesting cell wall components which are not separated from the soluble fraction. Examples of interesting components from the cell walls are β-glucans, mannoproteins and the yeast lipid fraction. These components may evoke or enhance mouthfeel attributes such as fattiness and fullness. Yeast autolysate has been known for many years as a source of protein, peptides, aminoacids such as cystein, fats, minerals and B-vitamins. A yeast autolysate rich in cystein would be a suitable cystein or glutathione source for producing process flavours. The yeast autolysate of the invention may be obtained from cream yeast or from the total fermentation broth, i.e. yeast cells including vinasse. Preferably the yeast autolysate of the invention has a dry solids ratio between 50 and 95. A process to produce a yeast autolysate with a dry solid ratio between 50 and 95 is described in WO2009/007424.

Preferably the amount of cystein in the yeast autolysate of the invention is at least 3.3 mg/g, more preferably at least 3.6 mg/g, 4.4 mg/ml even more preferably at least 4.8 mg/g based on total dry matter. Preferably the amount of 5′-ribonucleotides in the yeast autolysate of the invention is at least 2% w/w, 3%, 4%, more preferably at least 6, 8, 10% w/w, even more preferably at least 12%, 14%, 16%, even more preferably at least 18%, 20%, 22%, most preferably at least 25% w/w based on NaCl free dry matter weight, whereby NaCl free is defined as above.

Cystein and glutathione may be measured by several methods, for example by using NMR, liquid chromatography (LC), for example high-pressure LC (HPLC), or LC combined with mass spectrometry (LCMS), or (HP) LC-MSMS.

Cystein may also be determined using ninhydrin as described by M. K. Gaitonde, Biochemical Journal (1967), vol. 104, p. 627-633.

Cystein and/or glutathione may be recovered by techniques known in the art. For example, the cystein and/or glutathione may be recovered by centrifugation, whereby the microorganism is discarded as the pellet and the cystein and/or glutathione is recovered in the supernatant, or by filtration whereby the microorganism is discarded as the retentate (or filter cake) and the cystein and/or glutathione is recovered in the filtrate. When the cystine is contacted with a live microorganism or a killed but intact microorganism, prior to recovering the cystein and/or glutathione it may be preferred to lyse the microbial cell in order to release the cystein and/or glutathione. Cystein and/or glutathione may be recovered from a lysed microorganism suspension which comprises a solid fraction which mainly consists of cell walls for example by centrifugation and (ultra)filtration. The skilled person will understand that said solid fraction comprising cell walls may also comprise some cystein or glutathione. Therefore, it may be preferred to wash the solid fraction one or more times in order to recover as much cystein and glutathione as possible.

### EXAMPLES

### Example 1

### Cystein Production During Yeast Autolysis

Cream yeast from Saccharomyces cerevisiae (dry solids is 18.2%) was autolysed at pH 5.9 and 51° C. by adding endo-protease from Bacillus licheniformis (Alcalase, Novozymes, Denmark). During the autolysis cystine (2% w/w), reduced nicotinamide adenine dinucleotide (NADH, 1% w/w) and/or glucose (1% w/w) were added, either at the start of the autolysis (t=0 hrs) or 4 hours after the start of the autolysis (t=4 hrs), additions in w/w all based on total dry weight, according to Table 1.

After 20 hours of autolysis a solid liquid separation was done by centrifugation, without pH adjustment. The yield on dry matter was 70%. The supernatants, containing the soluble components from the yeast cells, were analysed for cystein content (mg/g dry matter) using NMR and HPLC. Results are presented in Table 1.

TABLE 1
Cystein content of the supernatants

| | | | | | Cystein |
|---|---|---|---|---|---|
| Experiment | Protease | Cystine | NADH | Glucose | (mg/g) |
| Comp. Ex. A | t = 0 hrs | — | — | — | 1.78 |
| Ex. 1 | t = 0 hrs | t = 4 hrs | t = 4 hrs | — | 5.05 |
| Ex. 2 | t = 4 hrs | t = 0 hrs | t = 0 hrs | — | 6.15 |
| Ex. 3 | t = 0 hrs | t = 0 hrs | t = 0 hrs | — | 4.63 |
| Ex. 4 | t = 0 hrs | t = 0 hrs | t = 0 hrs | t = 0 hrs | 6.74 |

### Example 2

### Cystein Production by Yeast Cell Walls and Yeast Extract

Approximately 2 kg of cream yeast from Saccharomyces cerevisiae (a yeast suspension with a dry solids content of 19.9% w/w) was heat-treated at 51° C. for 5 min. The pH of the suspension was subsequently adjusted to 6.0 using NaOH. The yeast suspension was autolysed by adding Bacillus subtilus serine endoprotease (obtained as Alcalase, Novozymes, Denmark) in an amount of 0.0068 g/g based on dry matter) and incubation for 3.5 h, resulting in an autolysate. The pH of the autolysate was subsequently lowered to 5.1 using H₂SO₄ and the autolysate was incubated for approximately 18 h. Next, the autolysate was centrifuged at 4400 rpm for 15 min, resulting in a pellet comprising cell walls and a supernatant which is a yeast extract. The pellet was washed with cold water 2 times and resuspended in water and centrifuged under same conditions. The supernatant was discarded and the pellet comprising the cell walls was resuspended in water to a final concentration of cell walls in the suspension of 7.7% w/w based on total dry weight of the cell walls. The dry matter content of the yeast extract was 15% w/w.

To 150 gram of the yeast extract were added cystine (1.2 g), glucose (1.3 g), glucose dehydrogenase (10 U/g), and oxidized nicotinamide adenine dinucleotide (0.5 mM). To 150 gram of the cell wall suspension were added cystine (0.6 g), glucose (0.7 g), glucose dehydrogenase (10 U/g), and oxidized nicotinamide adenine dinucleotide (0.5 mM). See Table 2. The pH was adjusted to 6.5. The cell wall suspension and the yeast extract were then incubated at 35° C. for approximately 18 hours. Cystein was measured spectrophotometrically using the ninhydrin reagent as described by M. K. Gaitonde, Biochemical Journal (1967), vol. 104, p. 627-633.

Glucose dehydrogenase was obtained from Codexis, Inc, 200 Penobscot Drive, Redwood City, Calif. 94063, USA.

TABLE 2
Amount of cystein formed (mg)

| | Cell wall | Yeast |
|---|---|---|
| Sample | suspension | extract |
| Comparative example (no addition) | 0.83 | 0.91 |
| With added cystine, oxidized nicotinamide adenine | 1.8 | 1.62 |
| dinucleotide, glucose, and glucose dehydrogenase | | |

### Example 3

### Cystein Production During Fermentation

Saccaromyces cerevisiae was grown in 100 mL shake flasks on mineral media according to Table 3. Vitamins, cofactors, and trace elements were added separately.

TABLE 3
Media composition (in g/500 mL unless otherwise indicated).

| Compound | Amount |
|---|---|
| NH₄H₂PO₄ | 30 |
| MgCl₂•6aq | 2 |
| NH₄Cl | 8.1 |
| KH₂PO₄ | 5 |
| NaCl | 0.5 |
| CaCl₂ (1M) * | 4.5 ml |

* added from a 1M stock solution after sterilization of the media

The media was heat-sterilized for 180 minutes at 160° C. The incubation temperature was 30° C. and the fermentation time was 24 hours. Every flask was fermented in duplicate (A and B). Other conditions are listed in Table 4. Cystein and cystine were added according to Table 5.

TABLE 4
Fermentation conditions

| | |
|---|---|
| Baffle | no |
| Stopper | water lock |
| Stirrer speed | 100 rpm |
| Fermentation volume | 25 mL |

TABLE 5
Addition of cystine

| Flask nr | addition | amount added (mg/L) | |
|---|---|---|---|
| 1 | no addition | 1A | — |
| | | 1B | — |
| 2 | +cystine | 2A | 400 |
| | | 2B | 480 |

At the end of fermentation, the absorbance of the broth at 600 nm was measured as a measure of growth. After that, the broth was split in two portions. One portion was boiled for 10 minutes in order to release cystein and/or glutathione. This resulted in a cell suspension. The dry matter content of this cell suspension was determined by drying and subsequent weighing. The other portion was filtrated; the resulting retentate was discarded and a clear filtrate was obtained. Both the filtrate and the cell suspension were analyzed for cystein both at t=0 (after inoculation) and at t=24 hours (Table 6).

TABLE 6
Fermentation results

cystein

| Flask | A(600) | | t = 0 | t = 24 |
|---|---|---|---|---|
| 1A | 0.405 | Filtrate | − | − |
| | | cell suspension | − | − |
| 1B | 0.413 | filtrate | − | − |
| | | cell suspension | − | − |
| 2A | 1.124 | filtrate | − | − |
| | | cell suspension | − | + |
| 2B | 1.167 | filtrate | − | − |
| | | cell suspension | − | − |

“−” means not detectable (i.e. below the detection limit);
“+” means detectable cystein.

Cystein was determined using an HPLC-MSMS system (Waters) using a ZIC-HILIC column (Merck, Darmstadt, Germany). ¹³C labeled cystein as internal standard was used.

1. A process for production of cystein and/or glutathione from cystine comprising contacting said cystine with a microorganism and recovering said cystein and/or said glutathione.

2. The process according to claim 1, wherein said microorganism is yeast.

3. The process according to claim 1, wherein the microorganism is lysed and comprises a cell wall.

4. The process according to claim 1, wherein said microorganism is in a fermentation media.

5. The process according claim 4, wherein said cystine is added to said fermentation media.

6. The process according to claim 1 wherein a reductant and/or a cofactor is present.

7. The process according to claim 1 wherein at least one enzyme is present.

8. The process according to claim 7, wherein said enzyme is selected from the group consisting of protease, cystein reductase, hydrogenase, and lipoamide dehydrogenase.

9. A yeast extract comprising at least 1.8 mg/g cystein based on total dry matter.

10. The yeast extract according to claim 9, comprising at least 1% w/w 5′-ribonucleotides based on NaCl free dry matter weight.

11. A yeast autolysate comprising at least 1.3 mg/g w/w cystein based on total dry matter.

12. The yeast autolysate according to claim 11, comprising at least 1% w/w 5′-ribonucleotides based on NaCl free dry matter weight.
